(19) United States
(12) Patent Application Publication
(10) Pub. No.: US 2006/0269952 A1
(43) Pub. Date: Nov. 30, 2006

(54) Genotyping method using distance measure

(76) Inventor: Ji-young Oh, Suwon-si (KR)

Correspondence Address: CANTOR COLBURN, LLP, 55 GRIFFIN ROAD SOUTH, BLOOMFIELD, CT, 06002, US

(21) Appl. No.: 11/440,847

(22) Filed: May 25, 2006

(30) Foreign Application Priority Data
May 27, 2005 (KR) 10-2005-0045216

(51) Int. Cl. C12Q 1/68; G06F 19/00

(52) U.S. Cl. 435/6; 702/20

(57) ABSTRACT

A genotyping method includes: (a) hybridizing a known standard nucleic acid to a DNA chip on which an optimal probe set composed of two or more different probes matching respective two or more different genotypes is immobilized for each mutation site, calculating an input vector having two components from the hybridization data, and setting up a genotyping algorithm using the input vector; (b) determining the centroid point of each of the two or more different genotypes; and (c) hybridizing an unknown target nucleic acid to the DNA chip, calculating an input vector having two components from the hybridization data, inputting the input vector into the genotyping algorithm, calculating a distance between the input vector and the centroid point of each of the two or more different genotypes, and determining that the target nucleic acid belongs to a genotype whose centroid point is nearest to the input vector for the target nucleic acid. Therefore, it can be determined that an unknown target nucleic acid belongs to which one of two or more genotypes, and in particular, to three or more genotypes.

This application claims priority to Korean Patent Application No. 10-2005-0045216, filed on May 27, 2005, and all the benefits accruing therefrom under 35 U.S.C. § 119, and the contents of which in its entirety are herein incorporated by reference.

### BACKGROUND OF THE INVENTION

1. Field of the Invention

The present invention relates to a genotyping method using a distance measure to determine that a target nucleic acid whose genotype is unknown belongs to which one of two or more genotypes, and in particular, to three or more genotypes.

2. Description of the Related Art

A typical genotyping method identifies sequences using a sequencing machine. This method is accurate but has low efficiency since this method is not suitable for simultaneous analysis of several samples.

Unlike the above method, DNA chips capable of simultaneously determining various genotypes at several sites are disclosed in U.S. Pat. Nos. 6,027,880 and 6,300,063. The DNA chips disclosed in the patents utilize tiled arrays of 9 to 25-mer oligonucleotide probes that vary at only a nucleotide position corresponding to a mutation site of a target sequence.

In other words, to achieve genotyping, together with sequencing, all possible base combinations are used for a tiled array of probes that are complementary to nucleotides at and near mutation sites. Thus, the number of required probes increases four times whenever one more tiled array site is required. However, such a tiled array includes redundant probes for an identified target nucleic acid. In addition, the tiled array method cannot be applied to mutations by insertion or deletion.

According to the tiled array method, numerous probes having a fixed length are used. These probes vary only at a nucleotide position corresponding to a particular locus, and thus have very similar sequences. Therefore, it is difficult to interpret the genotyped results for a particular locus, and the manufacturing costs of DNA chips increase. For example, if the hybridization intensity of a probe that perfectly matches a wild type gene (wild type-perfect match probe) or a probe that perfectly matches a mutant gene (mutant type-perfect match probe) is lower than the hybridization intensity of the other mismatch probes, a genotyping error occurs, which makes it difficult to prove a cross-hybridization effect. Also, the fixed length of the probes in the tiled array hinders optimal hybridization with a particular nucleic acid.

In view of the problems of the tiled array method, a genotyping method is disclosed in Korean Patent Application No. 2003-05025. The genotyping method includes setting up a genotyping algorithm using data obtained from hybridization of a known standard nucleic acid to a DNA chip, and determining the genotype of an unknown target nucleic acid by substituting an input vector, which is calculated from data obtained from hybridization of the unknown target nucleic acid to the DNA chip, into the genotyping algorithm. Posterior probabilities that the target nucleic acid belongs to each of two genotypes are calculated by substituting an input vector into the genotyping algorithm and it is determined that the target nucleic acid belongs to the genotype having greater posterior probability.

However, the genotyping method disclosed in Korean Patent Application No. 2003-05025 is a one-dimensional method dependent on a single parameter. Thus, it is possible to determine that a target nucleic acid belongs to which one of two genotypes, e.g., wild-type and mutant-type, but it is impossible to determine that a target nucleic acid belongs to which one of three or more genotypes.

### BRIEF SUMMARY OF THE INVENTION

While searching for solutions to the problems associated with the above conventional methods, the present inventor found a genotyping method capable of determining which one of three or more genotypes that a target nucleic acid belongs to by setting up a genotyping algorithm, inputting an input vector having two components into the genotyping algorithm, and calculating a distance between the input vector and the centroid point of each of the three or more genotypes.

Therefore, the present invention provides a genotyping method capable of determining which one of three or more genotypes that a target nucleic acid belongs to.

According to an exemplary embodiment of the present invention, a genotyping method includes: (a) hybridizing a known standard nucleic acid to a DNA chip on which an optimal probe set composed of two or more different probes matching respective two or more different genotypes is immobilized for each mutation site, calculating an input vector having two components from the hybridization data, and setting up a genotyping algorithm using the input vector; (b) determining the centroid point of each of the two or more different genotypes; and (c) hybridizing an unknown target nucleic acid to the DNA chip, calculating an input vector having two components from the hybridization data, inputting the input vector into the genotyping algorithm, calculating a distance between the input vector and the centroid point of each of the two or more different genotypes, and determining that the target nucleic acid belongs to a genotype whose centroid point is nearest to the input vector for the target nucleic acid.

### BRIEF DESCRIPTION OF THE DRAWINGS

The above and other features and advantages of the present invention will become more apparent by describing in detail exemplary embodiments thereof with reference to the attached drawings in which:

FIG. 1 is a flowchart illustrating an exemplary embodiment of a genotyping method according to the present invention;

FIG. 2 is a detailed flowchart for screening of an optimal probe set;

FIG. 3 is a detailed flowchart for setting up of a genotyping algorithm;

FIG. 4 is a detailed flowchart for genotyping;

FIG. 5 is a plot of a ratio component (M) versus an intensity component (A) (MA plot) used for setting up a genotyping algorithm for position MZA2415 of maize lines B73, MO17, and a hybrid thereof;

FIG. 6 is the MA plot of FIG. 5 in which the centroid points of the maize lines B73, MO17, and the hybrid are further plotted;

FIG. 7 is the MA plot of FIG. 6 in which the genotyped result for an unknown target nucleic acid is further plotted; and

FIG. 8 is the MA plot of FIG. 7 in which distances between the genotyped result for the unknown target nucleic acid and the centroid points of the maize lines B73, MO17, and the hybrid are further plotted.

### DETAILED DESCRIPTION OF THE INVENTION

Hereinafter, the present invention will be described more fully with reference to the accompanying drawings, in which exemplary embodiments of the invention are shown. This invention may, however, be embodied in many different forms and should not be construed as limited to the exemplary embodiments set forth herein. Rather, these embodiments are provided so that this disclosure will be thorough and complete, and will fully convey the scope of the invention to those skilled in the art. In the drawings, lengths and sizes of layers and regions may be exaggerated for clarity. Like numbers refer to like elements throughout. As used herein, the term “and/or” includes any and all combinations of one or more of the associated listed items.

It will be understood that, although the terms first, second, third, etc., may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, the term “DNA chip” refers to a microarray of a large number of nucleic acid probes. The term “nucleic acid” refers to nucleotides composed of pyrimidine bases, including cytosine (C) and guanine (G), and purine bases, including thymine (T) or uracil (U) and adenine (A), or polymers (polynucleotides) or oligomers (oligonucleotides) of the nucleotides. Examples of DNA chips include cDNA chips with at least 500 bp probes and oligonucleotide chips with 9 to 25-mer oligonucleotide probes. The term “standard nucleic acid” refers to a nucleic acid whose genotype is known. The term “target nucleic acid” refers to a nucleic acid of interest that has an unknown genotype. The target nucleic acid may be an oligonucleotide or polynucleotide of RNA or DNA. The term “probe” refers to a nucleic acid used to determine the genotype of the target nucleic acid.

In the flowcharts, blocks outlined by dashed lines denote optional operations.

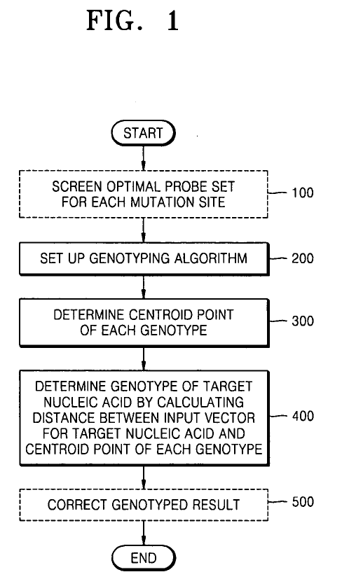

FIG. 1 is a flowchart illustrating an exemplary embodiment of a genotyping method according to the present invention.

Referring to FIG. 1, an exemplary embodiment of a genotyping method according to the present invention includes setting up a genotyping algorithm (operation 200), determining a centroid point of each of two or more genotypes (operation 300), and determining the genotype of a target nucleic acid by calculating a distance between an input vector for the target nucleic acid and the centroid point of each of the two or more genotypes (operation 400). Optionally, the genotyping method may further include screening an optimal probe set (operation 100) before operation 200 and correcting the genotyped results (operation 500) after operation 400.

In an exemplary embodiment according to the present invention, the genotyping method is used to determine the genotype of a target nucleic acid. In the genotyping method, a DNA chip, in which only an optimal probe set composed of two or more different probes perfectly matching respective two or more different genotypes, is immobilized for each mutation site,. Therefore, there is no need to immobilize unnecessary probes on the DNA chip. In addition, interpretation of the results is simple, errors resulting from cross-hybridization can be easily corrected, and the manufacturing costs of the DNA chip can be decreased. An exemplary embodiment of a genotyping method according to the present invention can also be applied to mutations by insertion or deletion.

Hereinafter, an exemplary embodiment of a genotyping method according to the present invention will be described step by step in more detail.

Screening of Optimal Probe Set for each Mutation Site

Referring to FIG. 1, an exemplary embodiment of a genotyping method of the present invention may further include screening an optimal probe set composed of two or more different probes perfectly matching respective two or more different genotypes for each mutation site (operation 100).

When there is a known optimal probe set for each mutation site, the screening of an optimal probe set for each mutation site may be omitted.

On the other hand, when an optimal probe set for each mutation site is unknown, the screening of the optimal probe set is performed. A method of designing an optimal probe set for each mutation site of an identified genotype is well known in the art.

For example, an optimal probe set for each mutation site can be designed by identifying the sequences of two or more different genotypes and preparing oligonucleotides perfectly hybridizing with the sequences of the two or more different genotypes.

An optimal probe set for each mutation site can also be screened by a modification of a method disclosed in Korean Patent Application No. 2003-05025, filed on Jan. 25, 2003, by the same applicant as the present application, the disclosure of which in its entirety is herein incorporated by reference.

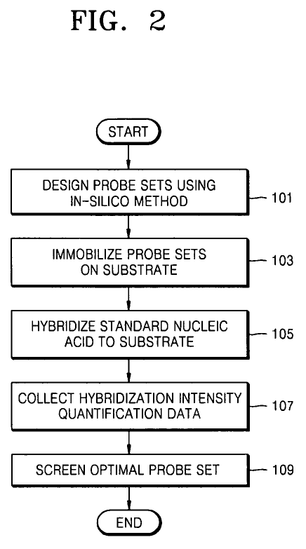

FIG. 2 is a detailed flowchart for operation 100 of FIG. 1. The screening of the optimal probe set illustrated in FIG. 2 is as described in Korean Patent Application No. 2003-05025.

Referring to FIG. 2, a plurality of probes complementary to each of two or more different genotypes for each mutation site are designed using an in-silico method (sub-operation 101). The plurality of the probes complementary to each of the two or more different genotypes may be the same or different in length. That is, there is no limitation to the length of the probes provided that the probes are complementary to the same strand. Then, all possible combinational sets of the probes complementary to the two or more different genotypes are immobilized on a substrate to complete an optimal probe set screening chip (sub-operation 103). The immobilization of the sets of the probes complementary to the two or more different genotypes on the substrate can be achieved by one of various methods known to those of ordinary skill in the art. For example, the probe sets can be immobilized on a chip according to a method disclosed in Korean Patent Application No. 2001-53687 filed by the same applicant as the present application, the disclosure of which in its entirety is herein incorporated by reference.

Next, a standard nucleic acid is hybridized to the optimal probe set screening chip (sub-operation 105). At this time, the hybridization is performed on a plurality of optimal probe set screening chips. The hybridization is performed by one of various methods known to those of ordinary skill in the art. After the hybridization is completed, hybridization intensity quantification data are collected by a scanner (sub-operation 107). A number of hybridization intensity quantification data are collected using the plurality of the optimal probe set screening chips. Finally, an optimal probe set for each mutation site is screened based on the hybridization intensity quantification data (sub-operation 109). A probe set having the greatest hybridization intensity is selected as an optimal probe set for each mutation site, which is within the ordinary knowledge of those of ordinary skill in the art and thus can be easily modified by those of ordinary skill in the art.

An optimal probe set for each mutation site can also be screened by a method disclosed in Korean Patent Application No. 2002-11871, filed on Mar. 6, 2002, by the same applicant as the present application, the disclosure of which in its entirety is herein incorporated by reference.

Setting Up of Genotyping Algorithm

Referring again to FIG. 1, an exemplary embodiment of a genotyping method of the present invention includes setting up the genotyping algorithm using a known optimal probe set or the above-screened optimal probe set composed of two or more different probes perfectly matching respective two or more different genotypes for each mutation site (operation 200).

The two or more different genotypes may be three or more different genotypes. For example, the two or more different genotypes may be three different genotypes including a wild-type gene, another wild-type gene and a hybrid gene thereof. Of course, the two or more different genotypes may be four or more different genotypes.

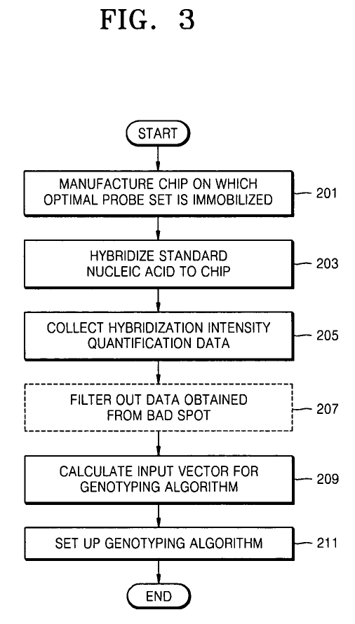

FIG. 3 is a detailed flowchart for the setting up of the genotyping algorithm (operation 200) of FIG. 1.

Referring to FIG. 3, first, a DNA chip is manufactured by arranging an optimal probe set for each mutation site in a microarray (sub-operation 201). The DNA chip can be manufactured in the same manner as described above in the manufacturing of the optimal probe set screening chip. It is preferable that at least two identical optimal probe sets are arranged for each mutation site in terms of quality control (“QC”) and quality assurance (“QA”). It is more preferable that at least two identical probes perfectly matching one genotype are arranged and at least two identical probes perfectly matching another genotype are arranged adjacent to the at least two identical probes perfectly matching the one genotype for each mutation site to visually detect the hybridized results. It is most preferable that three identical optimal probe sets are arranged for each mutation site in terms of QC, QA and costs. For example, in the case of identifying three different genotypes, e.g., a first wild-type gene, a second wild-type gene and a hybrid gene thereof, three identical probes perfectly matching the first wild-type gene are arranged, three identical probes perfectly matching the second wild-type gene are arranged adjacent to the three identical probes perfectly matching the first wild-type gene, and three identical probes perfectly matching the hybrid gene are arranged adjacent to the three identical probes perfectly matching the second wild-type gene.

Next, a standard nucleic acid is hybridized to the DNA chip (sub-operation 203) and hybridization intensity quantification data are then collected (sub-operation 205). The DNA chip is washed after the hybridization and the hybridization intensity quantification data are collected by a scanner.

Optionally, data obtained from bad spots among the hybridization intensity quantification data may be filtered out (sub-operation 207). Criteria for bad spot discrimination include an effective spot diameter cutoff value, an effective spot intensity cutoff value, etc., which are calculated based on a number of statistical data. In an exemplary embodiment of the present invention, spots that have a larger diameter than an effective spot diameter are regarded as bad spots and eliminated during statistical data processing.

Next, a vector for the genotyping algorithm is calculated using the hybridization intensity quantification data (sub-operation 209). The vector may be calculated using Hodge-Lehman (“H-L”) estimation that is typically applied in nonparametic statistics to raise the robustness of the genotyping algorithm. The vector used to set up the genotyping algorithm in the present invention includes a ratio component and an intensity component.

The ratio component is calculated by calculating all possible combinational ratios between the hybridization intensity of the standard nucleic acid to a probe perfectly matching one of two or more different genotypes and the hybridization intensity of the standard nucleic acid to a probe perfectly matching another one of the two or more different genotypes, selecting the median among the ratios, and calculating the logarithm of the median.

In more detail, all possible combinational ratios between the hybridization intensity of the standard nucleic acid to a probe perfectly matching one of two or more different genotypes and the hybridization intensity of the standard nucleic acid to a probe perfectly matching another one of the two or more different genotypes are calculated as expressed by Equation 1 below:
rᵢⱼ=(hybridization intensity to probe perfectly matching one genotype/hybridization intensity to probe perfectly matching another genotype), (1)

After calculating all possible ratios rᵢⱼ, the ratios rᵢⱼ are arranged in ascending order, for example, r(1)≦r(2) . . . ≦r(n−1)≦r(n), and the median, r(m), is selected among the ratios.

For example, when three identical probes perfectly matching a wild-type gene and three identical probes perfectly matching another wild-type gene are arranged for each mutation site, nine possible ratios rᵢⱼ are calculated and arranged in ascending order, i.e., r(1)≦. . . ≦r(5)≦. . . ≦r(9), and r(5) is selected as the median r(m).

The natural logarithm (In) of the median r(m) is used as a ratio component M, as expressed in Equation 2 below.
M=ratio component=In(r(m)), (2)

In some cases, the common logarithm (log) of the median r(m) instead of the natural logarithm (In) may be used as the ratio component.

The use of the median makes a genotyping algorithm more robust to experimental errors than using the arithmetic means of the hybridization intensities of identical probes.

Meanwhile, the intensity component is calculated by calculating all possible combinational maximum values of the hybridization intensities of the standard nucleic acid to two or more different probes perfectly matching respective two or more different genotypes, selecting the median among the maximum values, and calculating the logarithm of the median.

In more detail, all possible combinational maximum values of the hybridization intensities of the standard nucleic acid to two or more different probes perfectly matching respective two or more different genotypes are calculated. For example, all possible combinational maximum values of the hybridization intensities of a standard nucleic acid to three different probes perfectly matching respective three different genotypes are calculated as expressed by Equation 3 below:
mᵢⱼₖ=max(hybridization intensity to probe perfectly matching a wild-type gene, hybridization intensity to probe perfectly matching another wild-type gene, hybridization intensity to probe perfectly matching their hybrid gene), (3)

The median m(m) is selected from all of the maximum values mᵢⱼₖ and the common logarithm (log) of the median m(m) is used as an intensity component A, as expressed in Equation 4 below:
A=intensity component=log(m(m)), (4)

In some cases, the natural logarithm (In) of the median m(m) instead of the common logarithm (log) may be used as the intensity component.

Sub-operations 203 through 209 are performed using a plurality of chips to obtain a plurality of ratio components M and intensity components A. Again, it is noted that sub-operation 207 is optional, as indicated by the dashed lines in FIG. 3.

The genotyping algorithm is set up using vectors consisting of ratio (M) and intensity (A) components which are obtained based on the hybridization intensity quantification data according to the above-described methods (sub-operation 211).

To set up the genotyping algorithm, it is necessary to construct an MA plot with the y- and x-axes parameterized by the ratio (M) and intensity (A) components, respectively.

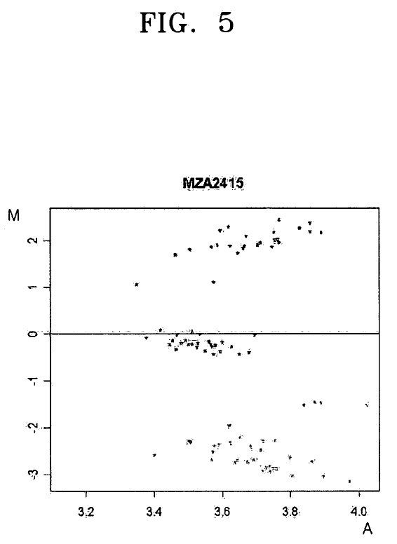

FIG. 5 is a MA plot used for setting up a genotyping algorithm for position MZA2415 of maize.

In hexaploid (6n) maize, a number of mutation or polymorphic sites are known. New maize lines with good character have been developed by artificially modifying the mutation or polymorphic sites. For example, a number of maize lines, B14, B37, B73, B84, MO17, etc. were developed. However, good character of the first generation of the maize lines may not be transmitted to subsequent generations of the maize lines, and a specific chromosomal site of the subsequent generations of the maize lines may have a hybrid genotype different from the genotype of the first generation of the maize species. Thus, the identification of a genotype at each mutation or polymorphic site enables determination that target maize belongs to which species or if it is a hybrid species different from an original single species.

The MA plot of FIG. 5 was obtained through the following processes.

First, an array of probes were immobilized on a glass substrate to manufacture a chip in which three identical optimal probes for the position MZA2415 of maize line B73 were arranged, three identical optimal probes for the position MZA2415 of maize line MO17 were arranged adjacent to the three identical optimal probes for the position MZA2415 of the maize line B73, and three identical optimal probes for the position MZA2415 of a hybrid of the maize lines B73 and MO17 were arranged adjacent to the three identical optimal probes for the position MZA2415 of the maize line MO17. A spotting solution obtained by mixing the probes with amine groups and hydrogels prepared from polyethyleneglycol (PEG) derivatives with epoxy groups was used to manufacture the chip. The spotting solution was spotted onto an aminated surface of the glass substrate using a biorobot printer (e.g., Model PixSys 5500, Cartesian Technologies Inc., CA, U.S.A.) and incubated in a humid incubator at 37° C. for 4 hours. To control background noise, amine groups in a non-spotting region of the glass substrate were negatively charged to prevent standard nucleic acids from binding to the non-spotting region of the glass substrate, and the glass substrate was then stored in a drier.

The standard nucleic acids were labeled with a fluorescent material. Available fluorescent materials include, for example, fluorescein isothiocyanate (FITC), fluorescein, Cy3, Cy5, Texas Red, and the like. In the experiment regarding the MA plot of FIG. 5, Cy3-dUTP was used as the fluorescent material.

The hybridization conditions between the standard nucleic acids and the probes were as follows. The chip was incubated in a solution of a 20 nM standard nucleic acid in 0.1% 6SSPET (saline sodium phosphate EDTA buffer containing 0.1% Triton X-100) at 37° C. for 16 hours, washed with 0.05% 6SSPET and 0.05% 3SSPET (5 minutes for each) at room temperature, dried at room temperature for 5 minutes, and scanned using an Axon scanner (Model GenePix 4000B, Axon Instrument Inc., CA., U.S.A.). The resulting scanning data were analyzed using a GenePix Pro 3.0 program (e.g., Axon Instrument Inc., CA., U.S.A.) to calculate ratio and intensity components to thereby obtain the MA plot of FIG. 5.

The genotyping algorithm may be set up using logistic regression coefficients (a, b) predicted by logistic regression.

Referring to FIG. 5, members belonging to the hybrid of the maize lines B73 and MO17 are represented by spots at the ratio component (M)=zero, members belonging to the maize line B73 are represented by spots at M>zero, and members belonging to the maize line MO17 are represented by spots at M<zero.

Determination of Centroid Points of Genotypes

Referring again to FIG. 1, after the setting up of the genotyping algorithm (operation 200) is completed, the centroid point of a genotype is determined (operation 300).

The centroid point of a genotype may be determined by calculating the medians of two components, i.e., ratio component (M) and intensity component (A), of each spot belonging to the genotype.

That is, when the MA plot coordinates of spots belonging to a genotype are G1(A1, M1), G2(A2, M2), G3(A3, M3),..., Gn(An, Mn), the centroid point of the genotype is calculated by Equation 5 below:
Centroid point=Gc(Ac, Mc)=(median(G1(A1), G2(A2), G3(A3), . . . , Gn(An)), median(G1(M1), G2(M2), G3(M3), . . . , Gn(Mn))), (5)

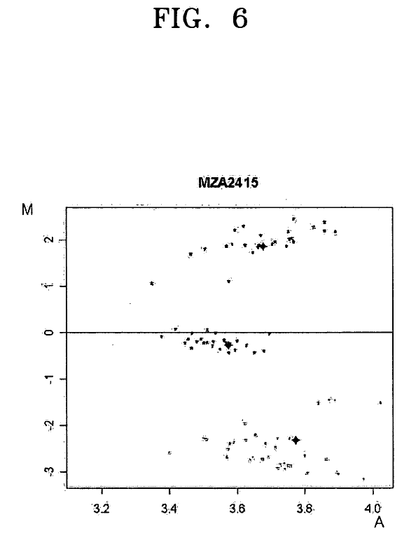

FIG. 6 is the MA plot of FIG. 5 in which the centroid point of each of the maize lines B73, MO17 and the hybrid is further plotted. Rhombohedrons in the MA plot of FIG. 6 represent the centroid points of the maize lines B73, MO17 and the hybrid. A rhombohedron is basically a “squashed” cube (e.g., truncated at the upper vertex).

Genotyping

Referring again to FIG. 1, after the genotyping algorithm is set up (operation 200) and the centroid points of the two or more genotypes are determined (operation 300) as described above, genotyping for an unknown target nucleic acid is performed (operation 400).

The genotyping for the target nucleic acid (operation 400) is achieved by calculating an input vector using test results obtained by applying the target nucleic acid to the DNA chip, inputting the input vector into the genotyping algorithm obtained in operation 200, calculating a distance between the input vector and the centroid point of each of the two or more genotypes, and determining that the target nucleic acid belongs to a genotype whose centroid point is nearest to the input vector for the target nucleic acid.

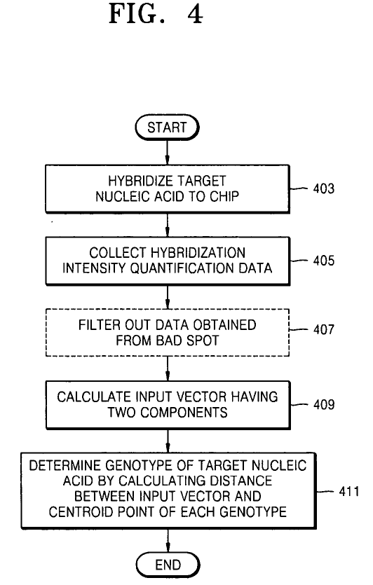

FIG. 4 is a detailed flowchart for the genotyping (operation 400) of FIG. 1.

Referring to FIG. 4, sub-operation 403 to sub-operation 409 is performed in the same manner as in operation 200 of FIG. 1 or sub-operations 201 to 209 of FIG. 3. First, a target nucleic acid is hybridized to the chip with which the genotyping algorithm has been set up (sub-operation 403). Then, hybridization intensity quantification data regarding the target nucleic acid are collected (sub-operation 405). Optionally, data obtained from bad spots may be filtered out from the hybridization intensity quantification data (sub-operation 407).

Next, an input vector for genotyping is calculated based on the hybridization intensity quantification data (sub-operation 409). Ratio and intensity components are calculated using H-L estimation as described above in the setting up of the genotyping algorithm. That is, the ratio component is calculated by calculating all possible combinational ratios between the hybridization intensity of the target nucleic acid to a probe perfectly matching one of two or more different genotypes and the hybridization intensity of the target nucleic acid to a probe perfectly matching another one of the two or more different genotypes, selecting the median among the ratios, and calculating the logarithm of the median. The intensity component is calculated by calculating all possible combinational maximum values of the hybridization intensities of the target nucleic acid to the two or more different probes perfectly matching the respective two or more different genotypes, selecting the median among the maximum values, and calculating the logarithm of the median.

Finally, the input vector is input into the genotyping algorithm, a distance between the input vector and the centroid point of each of the two or more different genotypes is calculated, and it is determined that the target nucleic acid belongs to a genotype whose centroid point is nearest to the input vector (sub-operation 411). The genotyped results for the target nucleic acid and the standard nucleic acids may be plotted together on the same MA plot for comparative visual identification.

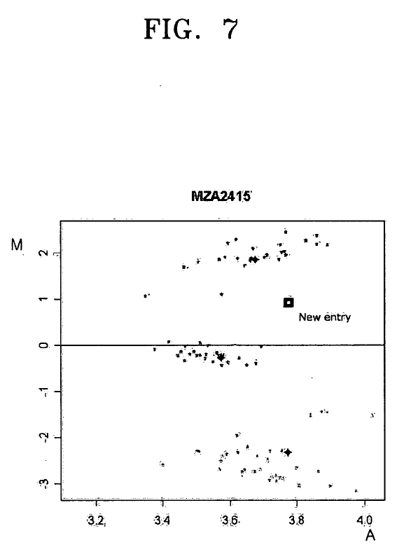

FIG. 7 is the MA plot of FIG. 6 in which the genotyped result for the unknown target nucleic acid is further plotted. Referring to FIG. 7, the genotyped result of the target nucleic acid is represented with a square and identified with a designation of “New entry”. In this case, it must be determined that the target nucleic acid belongs to which one of the three genotypes, i.e., B73, MO17 and the hybrid.

Genotyping is achieved based on a distance between the input vector for the target nucleic acid and the centroid point of each of the three genotypes. The distance between the input vector for the target nucleic acid and the centroid point of each of the three genotypes may be calculated using Euclidean distance. In detail, the Euclidean distance between the input vector for the target nucleic acid and the centroid point of each of the three genotypes is calculated using Equation 5 below:
Euclidean distance=[(Ac−Ax)²+(Mc−Mx)²]^{1/2}, (5)

where the centroid point of each of the three genotypes is Gc(Ac, Mc), and the input vector for the target nucleic acid is N(Ax, Mx).

It is determined that the target nucleic acid belongs to a genotype having a centroid point which is nearest to the input vector for the target nucleic acid.

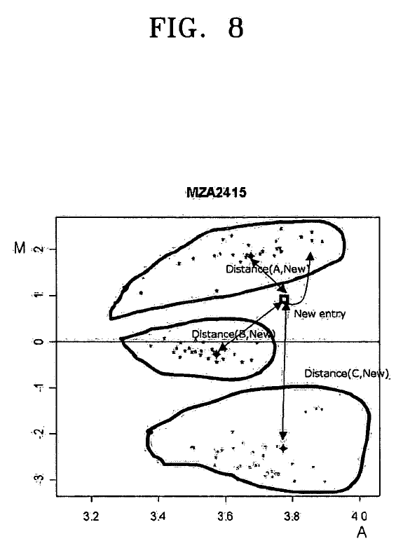

FIG. 8 is the MA plot of FIG. 7 in which distances between the input vector for the unknown target nucleic acid and the centroid points of the three genotypes, B73, MO17 and the hybrid, are further plotted.

Referring to FIG. 8, the input vector for the target nucleic acid is nearest to the centroid point of the maize line B73among the centroid points of the three genotypes, i.e., the maize lines B73, MO17 and the hybrid. Therefore, it can be determined that the genotype of the position MZA2415 of the target nucleic acid is B73.

If the degree of reliability on the distance at a predetermined significance level is not satisfied, the genotyping of the target nucleic acid may be deferred. The degree of reliability for the genotyping of the target nucleic acid is tested as follows. First, a confidence interval of the distance at a predetermined significance level is calculated. If 0.5 falls under the confidence interval, no genotyping of the target nucleic acid is performed (nocall). That is, the target nucleic acid is assigned as a gray zone. A method of calculating the confidence interval of the distance is described in detail in Chapter 1 of Applied Logistic Regression (Hosmer, D. W., Jr. and Lemeshow, S, John Wiley & Sons Inc., 1989), the disclosure of which in its entirety is herein incorporated by reference. To more strictly perform the genotyping, no genotyping is performed even when a value that is greater than 0.5, for example, 0.7, falls under the confidence interval. However, if the genotyping is deferred too frequently, the DNA chip does not work properly. Therefore, it is required to establish optimal genotyping criteria in consideration of the no-genotyping rate (nocall rate) and the mis-genotyping rate (miscall rate).

Correction of genotvped results Referring again to FIG. 1, after the genotyping is performed (operation 400) as described above, the genotyped results may be corrected (operation 500) to minimize nocall and miscall rates. The genotyped results can be corrected based on the result of cross-hybridization. For example, when it is known that a mutant type standard nucleic acid may be cross-hybridized with a probe set that is irrelevant to the identification of the mutation site of the standard nucleic acid, the genotyped results can be corrected using the cross-hybridization information on the standard nucleic acid.

The correction of the genotyped results is well known in the art. For example, the correction of the genotyped results can be performed using a method disclosed in Korean Patent Application No. 2003-05025, filed on Jan. 25, 2003, by the same applicant as the present application, the disclosure of which in its entirety is herein incorporated by reference.

As described above, a genotyping method of the present invention is a two-dimensional method using an input vector having two components. Therefore, the genotyping method of the present invention is more robust than a conventional one-dimensional genotyping method. In addition, the genotyping method of the present invention can also be applied in determining that a target nucleic acid belongs to which one of three or more different genotypes, unlike the conventional one-dimensional genotyping method.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A genotyping method comprising:
(a) hybridizing a known standard nucleic acid to a DNA chip on which an optimal probe set composed of two or more different probes matching respective two or more different genotypes is immobilized for each mutation site, calculating an input vector having two components from the hybridization data, and setting up a genotyping algorithm using the input vector;
(b) determining the centroid point of each of the two or more different genotypes; and
(c) hybridizing an unknown target nucleic acid to the DNA chip, calculating an input vector having two components from the hybridization data, inputting the input vector into the genotyping algorithm, calculating a distance between the input vector and the centroid point of each of the two or more different genotypes, and determining that the target nucleic acid belongs to a genotype whose centroid point is nearest to the input vector for the target nucleic acid.

2. The genotyping method of claim 1, wherein the two or more different genotypes are three or more different genotypes.

3. The genotyping method of claim 1, wherein the two or more different genotypes are three different genotypes comprising a first wild-type gene, a second wild-type gene and a hybrid gene of the first and second wild-type genes.

4. The genotyping method of claim 1, wherein operation (a) further comprises sub-operations (a-1 to a4), the sub-operations comprising:
(a-1) collecting hybridization intensity quantification data obtained by hybridizing the standard nucleic acid to the DNA chip;
(a-2) calculating a ratio component of the input vector for the standard nucleic acid by calculating all possible combinational ratios between the hybridization intensity of the standard nucleic acid to a probe matching one of the two or more different genotypes and the hybridization intensity of the standard nucleic acid to a probe matching another one of the two or more different genotypes, selecting the median among the ratios, and calculating the logarithm of the median;
(a-3) calculating an intensity component of the input vector for the standard nucleic acid by calculating all possible combinational maximum values of the hybridization intensities of the standard nucleic acid to the two or more different probes matching the respective two or more different genotypes, selecting the median among the maximum values, and calculating the logarithm of the median; and
(a-4) setting up the genotyping algorithm using sets of input vectors obtained by repeating sub-operations (a-1) through (a-3) using a plurality of DNA chips.

5. The genotyping method of claim 4, wherein in sub-operation (a4), logistic regression coefficients predicted by logistic regression are calculated using the sets of the input vectors.

6. The genotyping method of claim 4, wherein operation (a) further comprises setting the ratio component as an x-axis component and the intensity component as a y-axis component, prior to sub-operation (a-4).

7. The genotyping method of claim 4, wherein operation (a) further comprises filtering out hybridization intensity quantification data obtained from bad spots having a larger diameter than an effective spot diameter cutoff value among the hybridization intensity quantification data, prior to sub-operation (a-2).

8. The genotyping method of claim 1, wherein in operation (b), the medians of the two components are defined as the centroid point of each of the two or more different genotypes.

9. The genotyping method of claim 1, wherein operation (c) further comprises sub-operations (c-1 to c-4), the sub-operations comprising:
(c-1) collecting hybridization intensity quantification data obtained by hybridizing the target nucleic acid to the DNA chip;
(c-2) calculating a ratio component of the input vector for the target nucleic acid by calculating all possible combinational ratios between the hybridization intensity of the target nucleic acid to a probe matching one of the two or more different genotypes and the hybridization intensity of the target nucleic acid to a probe matching another one of the two or more different genotypes, selecting the median among the ratios, and calculating the logarithm of the median;
(c-3) calculating an intensity component of the input vector for the target nucleic acid by calculating all possible combinational maximum values of the hybridization intensities of the target nucleic acid to the two or more different probes matching the respective two or more different genotypes, selecting the median among the maximum values, and calculating the logarithm of the median; and
(c-4) inputting the input vector into the genotyping algorithm, calculating the distance between the input vector and the centroid point of each of the two or more different genotypes, and determining that the target nucleic acid belongs to a genotype whose centroid point is nearest to the input vector for the target nucleic acid.

10. The genotyping method of claim 9, wherein in sub-operation (c-4), the distance between the input vector and the centroid point of each of the two or more different genotypes is calculated using Euclidean distance.

11. The genotyping method of claim 9, wherein sub-operation (c-4) comprises:
inputting the input vector into the genotyping algorithm, calculating the distance between the input vector and the centroid point of each of the two or more different genotypes, and provisionally determining that the target nucleic acid belongs to a genotype whose centroid point is nearest to the input vector for the target nucleic acid; and
determining the degree of reliability on the distance at a predetermined significance level, and deferring genotyping of the target nucleic acid if the reliability requirement is not satisfied.

12. The genotyping method of claim 9, wherein operation (c) further comprises filtering out hybridization intensity quantification data obtained from bad spots having a larger diameter than an effective spot diameter cutoff value among the hybridization intensity quantification data, prior to sub-operation (c-2).

13. The genotyping method of claim 1, wherein at least two identical optimal probe sets are immobilized for each mutation site.

14. The genotyping method of claim 13, wherein the two or more different probes matching the respective two or more different genotypes are immobilized for each mutation site such that at least two identical probes matching one genotype are arranged and at least two identical probes matching another genotype are arranged adjacent to the at least two identical probes matching the one genotype.

15. The genotyping method of claim 1, wherein the optimal probe set for each mutation site is screened by:
designing a plurality of different probe sets, each of which is composed of two or more different probes matching respective two or more different genotypes, using an in-silico method;
immobilizing the plurality of the different probe sets on substrates to manufacture optimal probe set screening chips;
hybridizing the standard nucleic acid to the optimal probe set screening chips;
collecting hybridization intensity quantification data; and
screening a probe set having the greatest hybridization intensity.

16. The genotyping method of claim 1, further comprising correcting the genotyped results of operation (c) based on cross-hybridization data of the probe set for each mutation site.

17. The genotyping method of claim 1, wherein the optimal probe set composed of the two or more different probes matching respective two or more different genotypes perfectly matches the respective two or more different genotypes.
